(19) United States
(12) Patent Application Publication
(10) Pub. No.: US 2013/0059385 A1
(43) Pub. Date: Mar. 7, 2013

(54) METHODS OF GENERATING PLURIPOTENT STEM CELLS

(71) Applicants: Wenlin Li, San Diego, CA (US); Tongxiang Lin, San Diego, CA (US); Sheng Ding, Orinda, CA (US)

(72) Inventors: Wenlin Li, San Diego, CA (US); Tongxiang Lin, San Diego, CA (US); Sheng Ding, Orinda, CA (US)

(73) Assignee: THE SCRIPPS RESEARCH INSTITUTE, La Jolla, CA (US)

(21) Appl. No.: 13/582,011

(22) PCT Filed: Mar. 2, 2010
(86) PCT No.: PCT/US10/25915

§ 371 Date: Nov. 19, 2012

(51) Int. Cl. C12N 5/071; C09K 3/00; C12N 15/85

(52) U.S. Cl. 435/455; 435/377; 435/366; 252/182.29

(57) ABSTRACT

Disclosed are methods, compositions, and kits of producing an induced pluripotent stem cell from a mammalian non-pluripotent cell that does not endogenously or heterologously express Sox2 and is not in contact with a Sox2 polypeptide.

### BACKGROUND OF THE INVENTION

Pioneering work showed that virus-mediated expression of four transcription factors, Oct4, Klf4, Sox2 and c-Myc [Takahashi K, Yamanaka S, Cell, 126:663-676 (2006)], reprograms mouse somatic cells into induced pluripotent stem (iPS) cells which closely resemble embryonic stem (ES) cells [Takahashi K, Yamanaka S, Cell, 126:663-676 (2006); Okita et al., Nature, 448:313-317 (2007)]. Reprogramming human somatic cells had also been achieved through similar strategy [Takahashi et al, Cell, 131:861-872 (2007); Yu et al., Science, 318:1917-1920 (2007)]. The iPS cell technology has attracted enormous interests with respect to its potential practical applications. By reprogramming and differentiation processes, patient-specific pluripotent stem cells could be created and further differentiated into functional autologous cells for cell-based therapy with alleviated immunocompatibility issues and ethical concerns. However, iPS cell applications are hindered by safety concerns and its complexity as the generation of iPS cells typically involves integration of exogenous DNA sequences. The key advances aimed at overcoming these safety concerns have been achieved by using non-integrating gene delivery approaches (such as adenovirus or episomal plasmid transfection) [Stadtfeld et al., Science, 322:945-949 (2008); Okita et al., Science, 322:949-953 (2008); Yu et al., Science, 324:797-801 (2009)], or using cell membrane permeable proteins to trigger the reprogramming [Kim et al., Cell Stem Cell, 4:472-476 (2009); Zhou et al., Cell Stem Cell, 4:381-384 (2009)]. However, reprogramming is extremely slow and inefficient under such conditions, which presents significant hurdles and potential risks to generate human iPS cells. Identification of small molecules or novel conditions that can enhance reprogramming or compensate the requirement of certain reprogramming factors will be highly desirable. We and others have shown it is possible to generate iPS cells with fewer factors by exploiting the endogenous gene expression [Kim et al., Nature, 454:646-650 (2008); Shi et al., Cell Stem Cell., 2:525-528 (2008); Eminli et al., Stem Cells, 26:2467-2474 (2008)]. Neural progenitor cells (NPCs) with endogenous Sox2 expression [Blelloch et al., Stem Cells, 24:2007-2013 (2006)], could be reprogrammed into authentic iPS cells with only Oct4 and Klf4 transduction, however with a lower efficiency [Kim et al., Nature, 454:646-650 (2008); Shi et al., Cell Stem Cell., 2:525-528 (2008)]. Using a chemical screen, a G9a histone methyltransferase inhibitor, BIX-01294 [Kubicek et al., Mol Cell, 25:473-481 (2007)] was identified to enhance the reprogramming efficiency over 8 fold or replace the requirement of Oct4 transduction in NPC reprogramming [Shi et al., Cell Stem Cell., 2:525-528 (2008)]. BIX-01294 was also shown to enable the reprogramming of MEFs (which do not express Sox2) under Oct4 and Klf4 two factor conditions [Shi et al., Cell Stem Cell, 3:568-574 (2008)]. From a subsequent synergistic screen, other small molecules, e.g. DNA methyltransferase (DNMT) inhibitor RG108 and L-type calcium channel agonist BayK8644, were identified to enhance MEF reprogramming. Similarly, another DNMT inhibitor, 5-AZA, was shown to improve the reprogramming efficiency in MEF cells up to 4 folds by transiting partially reprogrammed cells to become fully pluripotent. In another study, histone deacetylase (HDAC) inhibitors such as valproic acid (VPA) were shown to be able to enhance the reprogramming efficiency [Huangfu et al., Nat Biotechnol., 26:795-797 (2008)]. In particular, VPA enabled reprogramming of human fibroblasts with only Oct4 and Sox2 [Huangfu et al., Nat Biotechnol., 26:1269-1275 (2008)].

Previous studies showed that Wnt3a conditioned media promotes reprogramming of MEF cells [Marson et al., Cell Stem Cell, 3:132-135 (2008)]. Wnt signaling entails inhibition of glycogen synthase kinase 3 (GSK-3) and stabilization of cytoplasmic β-catenin. Small molecule inhibitors of GSK-3 can mimic the activation of Wnt signaling, and maintain the pluripotent state of mES cells [Sato et al., Nat Med., 10:55-63 (2004); Bone et al., Chem Biol., 16:15-27 (2009); Umehara et al., Stem Cells, 25:2705-2711 (2007)]. Lluis F. et al.

reported that BIO, a GSK-3 inhibitor, could promote the reprogramming of somatic cells after fusion with mES cells [Lluis et al., Cell Stem Cell., 3:493-507 (2008)]. Silva et al. reported inhibition of MEK and GSK-3 (using PD0325901 and CHIR99021, respectively) could transit “pre-iPS cells” into fully reprogrammed pluripotent cells [Silva et al., PLoS Biol., 6:e253 (2008)]. More recently, Lyssiotis et al. identified another GSK-3/CDK2 inhibitor, kenpaullone, which could substitute Klf4 in reprogramming of MEFs in the presence of Oct4, Sox2 and cMyc. However, as a more specific GSK-3 inhibitor, CHIR99021, failed in producing the same positive effects on inducing the reprogramming of MEF cells under the Oct/Sox2/c-Myc transduction, kenpaullone's effect may not result from its GSK-3 inhibition and its precise mechanism remains elusive.

### BRIEF SUMMARY OF THE INVENTION

The present invention provides methods of producing an induced pluripotent stem cell from a mammalian non-pluripotent cell that does not endogenously or heterologously express Sox2 and is not in contact with a Sox2 polypeptide. In some embodiments, the method comprises,

introducing at least an Oct polypeptide and a Klf polypeptide into the non-pluripotent cell;
contacting the non-pluripotent cell with:
a GSK-3 inhibitor in the absence of an epigenetic modifier, or at least in the absence of an agent that inhibits histone H3K9 methylation or promotes H3K9 demethylation, or
a GSK-3 inhibitor and an inhibitor of histone H3K4 demethylation or an activator of H3K4 methylation,
under conditions to generate a pluripotent cell, thereby producing an induced pluripotent stem cell from the mammalian non-pluripotent cell.

In some embodiments, the method does not include contacting the cell with an agent that inhibits histone H3K9 methylation or promotes H3K9 demethylation.

In some embodiments, the Oct polypeptide comprises an Oct3/4 polypeptide.

In some embodiments, the Klf polypeptide comprises a Klf4 polypeptide.

In some embodiments, the GSK-3 inhibitor is CHIR99021.

In some embodiments, the Oct polypeptide comprises a human or mouse Oct polypeptide and the Klf polypeptide comprises a human or mouse Klf polypeptide.

In some embodiments, the introducing step comprises contacting the cells with the Oct polypeptide and the Klf polypeptide under conditions to allow for entry of the Oct polypeptide and the Klf polypeptide into the cell. In some embodiments, the Oct polypeptide comprises a heterologous amino acid sequence that enhances transport across a cell membrane; and the Klf polypeptide comprises a heterologous amino acid sequence that enhances transport across a cell membrane.

In some embodiments, the introducing step comprises contacting the cells with a polynucleotide encoding the Oct polypeptide and a polynucleotide encoding the Klf polypeptide under conditions to allow for (1) entry of the polynucleotide into the cell and (2) expression of the Oct polypeptide and the Klf polypeptide from the introduced polynucleotide.

In some embodiments, a heterologous Myc polypeptide is not introduced into, or contacted to, the non-pluripotent cell.

In some embodiments, the contacting step further comprises contacting the cell with an inhibitor of histone H3K4 demethylation or an activator of H3K4 methylation. In some embodiments, the inhibitor of histone H3K4 demethylation is a monoamine oxidase inhibitor. In some embodiments, the inhibitor of histone H3K4 demethylation is parnate.

In some embodiments described above, the contacting step further comprises contacting the cell with a Mek inhibitor and/or a TGFβ receptor inhibitor.

In some embodiments, the non-pluripotent cell is selected from a fibroblast and a keratinocyte.

In some embodiments, the cell is from human. In some embodiments, the cell is from a non-human mammal.

The present invention also provides cell culture media. In some embodiments, the medium comprises:

mammalian non-pluripotent cells that are not in contact with or express, Sox2;
an exogenous Oct polypeptide and an exogenous Klf polypeptide; and
a sufficient amount of:
a GSK-3 inhibitor in the absence of an epigenetic modifier, or
a GSK-3 inhibitor and an inhibitor of histone H3K4 demethylation or an activator of H3K4 methylation,
to induce at least 0.001% of the cells to pluripotency in the presence of the exogenous polypeptides.

In some embodiments, the culture does not include an agent that inhibits histone H3K9 methylation or promotes H3K9 demethylation.

In some embodiments, the Oct polypeptide comprises an Oct3/4 polypeptide.

In some embodiments, the Klf polypeptide comprises a Klf4 polypeptide.

In some embodiments, the GSK-3 inhibitor is CHIR99021.

In some embodiments, the Oct polypeptide comprises a human or mouse Oct polypeptide and the Klf polypeptide comprises a human or mouse Klf polypeptide.

In some embodiments, the Oct polypeptide comprises a heterologous amino acid sequence that enhances transport across a cell membrane; and the Klf polypeptide comprises a heterologous amino acid sequence that enhances transport across a cell membrane.

In some embodiments, a heterologous Myc polypeptide is not expressed in, or contacted to, the non-pluripotent cell.

In some embodiments, the medium further comprisea an inhibitor of histone H3K4 demethylation or an activator of H3K4 methylation. In some embodiments, the inhibitor of histone H3K4 demethylation is a monoamine oxidase inhibitor. In some embodiments, the inhibitor of histone H3K4 demethylation is a lysine-specific demethylase 1. In some embodiments, the inhibitor of histone H3K4 demethylation is parnate.

In some embodiments, the cell culture medium further comprises a Mek inhibitor and/or a TGFβ receptor inhibitor.

In some embodiments, the non-pluripotent cell is selected from a fibroblast and a keratinocyte.

In some embodiments, the cell is a human cell.

The present invention also provides kits. In some embodiments, the kit comprises an inhibitor of histone H3K4 demethylation or an activator of H3K4 methylation; and a GSK-3 inhibitor.

In some embodiments, the kit further comprises an Oct polypeptide and/or a Klf polypeptide.

In some embodiments, the GSK-3 inhibitor is CHIR99021.

In some embodiments, the Oct polypeptide comprises a heterologous amino acid sequence that enhances transport across a cell membrane; and the Klf polypeptide comprises a heterologous amino acid sequence that enhances transport across a cell membrane.

In some embodiments, the kit further comprises a Mek inhibitor and/or a TGFβ receptor inhibitor.

In some embodiments, the inhibitor of histone H3K4 demethylation is a monoamine oxidase inhibitor. In some embodiments, the inhibitor of histone H3K4 demethylation is a lysine-specific demethylase 1. In some embodiments, the inhibitor of histone H3K4 demethylation is parnate.

In some embodiments, the kit further comprises a mammalian cell. In some embodiments, the mammalian cell does not express Sox2.

This document incorporates by reference Li et al. Stem Cells 27(12):2992-3000 (2009).

Additional aspects of the invention will be clear from a reading of this entire document.

### DEFINITIONS

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include, Oct-1, Oct-2, Oct-3/4, Oct-6, Oct-7, Oct-8, Oct-9, and Oct-11. e.g. Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, or 95% amino acid sequence identity across their whole sequence compared to a naturally occurring Oct polypeptide family member such as to those listed above or such as listed in Genbank accession number NP_{—}002692.2 (human Oct4) or NP_{—}038661.1 (mouse Oct4). Oct polypeptides (e.g., Oct3/4) can be from human, mouse, rat, bovine, porcine, or other animals. Generally, the same species of protein will be used with the species of cells being manipulated.

A “Klf polypeptide” refers to any of the naturally-occurring members of the family of Krüppel-like factors (Klfs), zinc-finger proteins that contain amino acid sequences similar to those of the Drosophila embryonic pattern regulator Krüppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, Dang, D. T., Pevsner, J. & Yang, V. W., Cell Biol. 32,1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. See, Nakagawa, et al., Nature Biotechnology 26:101-106 (2007). In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated. To the extent a Klf polypeptide is described herein, it can be replaced with an estrogen-related receptor beta (Essrb) polypeptide. Thus, it is intended that for each Klf polypeptide embodiment described herein, a corresponding embodiment using Essrb in the place of a Klf4 polypeptide is equally described.

A “Myc polypeptide” refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals. Generally, the same species of protein will be used with the species of cells being manipulated.

A “Sox polypeptide” refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member , or polypeptides comprising at least the DNA-binding domain of the naturally occurring family member, and can further comprise a transcriptional activation domain. See, e.g., Dang, D. T., et al., Int. J. Biochem. Cell Biol. 32:1103-1121 (2000). Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. See, Nakagawa, et al., Nature Biotechnology 26:101-106 (2007). In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals. Generally, the same species of protein will be used with the species of cells being manipulated.

“H3K9” refers to histone H3 lysine 9. H3K9 modifications associated with gene activity include H3K9 acetylation and H3K9 modifications associated with heterochromatin, include H3K9 di-methylation or tri-methylation. See, e.g., Kubicek, et al., Mol. Cell 473-481 (2007). “H3K4” refers to histone H3 lysine 4. See e.g., Ruthenburg et al., Mol. Cell, 25:15-30 (2007).

The term “pluripotent” or “pluripotency” refers to cells with the ability to give rise to progeny that can undergo differentiation, under the appropriate conditions, into cell types that collectively demonstrate characteristics associated with cell lineages from all of the three germinal layers (endoderm, mesoderm, and ectoderm). Pluripotent stem cells can contribute to many or all tissues of a prenatal, postnatal or adult animal. A standard art-accepted test, such as the ability to form a teratoma in 8-12 week old SCID mice, can be used to establish the pluripotency of a cell population, however identification of various pluripotent stem cell characteristics can also be used to detect pluripotent cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics.

The term “library” is used according to its common usage in the art, to denote a collection of molecules, optionally organized and/or cataloged in such a way that individual members can be identified. Libraries can include, but are not limited to, combinatorial chemical libraries, natural products libraries, and peptide libraries.

A “recombinant” polynucleotide is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

“Expression cassette” refers to a polynucleotide comprising a promoter or other regulatory sequence operably linked to a sequence encoding a protein.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

A “heterologous sequence” or a “heterologous nucleic acid”, as used herein, is one that originates from a source foreign to the particular host cell, or, if from the same source, is modified from its original form. Thus, a heterologous expression cassette in a cell is an expression cassette that is not endogenous to the particular host cell, for example by being linked to nucleotide sequences from an expression vector rather than chromosomal DNA, being linked to a heterologous promoter, being linked to a reporter gene, etc.

The terms “agent” or “test compound” refer to any compound useful in the screening assays described herein. An agent can be, for example, an organic compound (e.g., a small molecule such as a drug), a polypeptide (e.g., a peptide or an antibody), a nucleic acid (e.g., DNA, RNA, double-stranded, single-stranded, an oligonucleotide, antisense RNA, small inhibitory RNA, micro RNA, a ribozyme, etc.), an oligosaccharide, a lipid. Usually, the agents used in the present screening methods have a molecular weight of less than 10,000 daltons, for example, less than 8000, 6000, 4000, 2000 daltons, e.g., between 50-1500, 500-1500, 200-2000, 500-5000 daltons. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., ability to induce pluripotency under certain conditions such as are described herein, creating variants of the lead compound, and evaluating the property and activity of those variant compounds. Often, high throughput screening (HTS) methods are employed for such an analysis.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

### BRIEF DESCRIPTION OF THE DRAWINGS

FIG. 1. CHIR99021 promoted the reprogramming of MEFs transduced by Oct4, Sox2 and Klf4. MEFs from 129 strain were transduced with Oct4, Sox2 and Klf4 by retroviruses, and treated the next day with increasing concentrations of CHIR99021 for two week. Three weeks later, AP was detected by staining cells in monolayer (A). ROSA26+/−/OG2+/−MEFs transduced with Oct4, Sox2 and Klf4 were seeded into 6-well plates at the density of 1×10⁴ cells/well (together with 10⁵ cells/well CF1 feeders) and treated with CHIR99021 for two weeks. After 3 weeks of treatment, the GFP positive colonies in each well were counted (B). Error bars represent standard deviation for N=3.

FIG. 2. CHIR99021 enable the reprogramming of MEFs transduced with Oct4 and Klf4 only. ROSA26+/−/OG2+/−MEFs transduced with Oct4 and Klf4 were splited into 6-well plates at the density of 10⁵ cells/well and treated with 10 μM CHIR99021 for 4 weeks. Panel A shows GFP-positive colonies before picking Total of four miPSCs-OK lines were established (B). The expression of Oct4 (C), Sox2 (D), Nanog (E) and SSEA-1(F) by miPSCs-OK was detected by immunocytochemistry. The expression of pluripotency genes by MEFs after CHIR99021 treatment was analyzed by real-time PCR. Scale bars, 20μm. Error bars represent standard deviation for N=3.

FIG. 3. PCR analysis and in vitro differentiation of miPSCs-OK. The expression of typical endogenous pluripotency genes and transduced genes (Tg) were analyzed by RT-PCR (A). Genomic PCR revealed integration of Oct4 and Klf4 retrovirues (B). 1˜4 referred to the four established miPSCs-OK lines. 100 bp DNA ladder (invitrogen) was used as a marker. Rat iPSCs generated by Oct4/Klf4/Sox2 transductions were used as positive control (+) and MEFs were used as negative control (−). Under standard EB differentiation methods, the in vitro pluripotency of miPSCs-OK was analyzed by Immunostaining (C-E). miPSCs-OK efficiently incorporated into the ICM of a blastocyst after aggregation with an 8-cell embryo (F). Chimeric embryo (13.5 dpc) was obtained after the transfer of the aggregated embryos into a pseudo-pregnant mouse. LacZ staining showed the contribution of miPSCs-OK (G). miPSCs-OK contributed to the germline cells (GFP-positive) in male gonad tissue isolated from chimeric embryos (I). Scale bars, 20 μm.

FIG. 4. hiPSCs-OK are generated from primary human keratinocytes transduced by Oct4 and Klf4. Established hiPSC-OK clones express pluripotency markers AP (A), SSEA3 (green)/Oct4 (red; B), TRA-1-81 (green)/Nanog (red; C), and SSEA4(green)/Sox2 (red; D). Expression of endogenous (endo) markers and viral transgenes in hiPS-OK 1, 2 and 3 was determined by real-time PCR (E). Primary human keratinocytes (K) and Hues9 human ES cells were used as controls. Error bars represent standard deviation for N=3. The methylation status of the Oct4 promoter in primary human keratinocytes and hiPSC-OK was analyzed using bisulfite sequencing. Open circles indicate unmethylated, and filled circles indicate methylated CpG dinucleotides (F).

FIG. 5. Real-time PCR analysis of the expression of pluripotency genes by keratinocytes after small molecule treatment. Primary human keratinocytes (K), keratinocytes treated with either combination of 10 μM CHIR99021 and 2 μM Parnate (2 inhibitors), or combination of 10 μM CHIR99021, 2 μM Parnate, 0.5 μM PD0325901, and 2 μM SB431542 (4 inhibitors), and Hues9 human ES cells were analyzed. Error bars represent standard deviation for N=3.

FIG. 6. hiPSCs-OK showed pluripotent potential in vitro and in vivo. Using the standard EB differentiation method, the in vitro pluripotency of hiPSCs-OK was analyzed by immunostaining (A-C). hiPSCs-OK generated full teratoma in SCID mice. Hematoxylin and eosin staining of hiPSCs-OK teratoma sections showed epithelial tube structure (endoderm), cartilage-like structure (mesoderm) and neuroepithelium-like structure (ectoderm) appear in (D-F). Scale bars, 20 μm.

### DETAILED DESCRIPTION

### I. Introduction

As presented in more detail in the Examples, the inventors have found, surprisingly, that it is possible to reprogram a somatic animal cell into an induced pluripotent stem cell in the absence (i.e., without endogenous or heterologous expression or introduction) of Sox2. Notably, the inventors have determined that reprogramming in the absence of Sox2 can be achieved by contacting the somatic cell with a GSK-3 inhibitor in the absence of an epigenetic modifier, or alternatively, with a GSK-3 inhibitor and an inhibitor of histone H3K4 demethylation. In some embodiments, the invention further involves introducing at least an Oct polypeptide and/or a Klf polypeptide into the cell. Introduction of the heterologous polypeptide(s) can be achieved, for example, by contacting the polypeptide(s) to the somatic cell and/or by introduction of a polynucleotide that encodes the polypeptide(s) into the cell under conditions such that the polypeptides(s) are expressed in the cell. Notably, the present invention provides evidence of histone H3K4 in reprogramming and the advantage of promoting methylation or inhibiting demethylation of H3K4.

### II. Reprogramming Factors

To date, a large number of different methods and protocols have been established for inducing non-pluripotent mammalian cells into induced pluripotent stem cells (iPSCs). iPSCs are similar to ESCs in morphology, proliferation, and pluripotency, judged by teratoma formation and chimaera contribution. The inventors have found that inclusion of a GSK3 inhibitor (in the absence of an epigenetic modifier, or optionally in the presence of a histone H3k4 demethylase inhibitor) in combination with introduction of an Oct and Klf polypeptide is effective in generating iPSCs in somatic animal cells that do not express Sox2. It is believed that the agents described herein (e.g., GSK3 inhibitors, optionally in combination with an agent that promotes histone H3K4 methylation or inhibits H3K4 demethylation) can be used in combination with essentially any protocol for generating iPSCs. In some embodiments, the agents described herein are used in combination with a reprogramming protocol that comprises introduction of either an Oct polypeptide, a Klf polypeptide, or both into a cell that does not express Sox2, thereby generating an induced pluripotent stem cell. In some embodiments, the protocol does not involve introduction of a Myc polypeptide and/or a Sox polypeptide.

In some embodiments, the reprogramming protocols will be a modified version of those previously described, but, for example, omitting c-Myc and/or Sox2. Studies have shown that retroviral transduction of mouse fibroblasts with four transcription factors that are highly expressed in ESCs (Oct-3/4, Sox2, KLF4 and c-Myc) generate induced pluripotent stem (iPS) cells. See, Takahashi, K. & Yamanaka, S. Cell 126, 663-676 (2006); Okita, K., Ichisaka, T. & Yamanaka, S. Nature 448, 313-317 (2007); Wernig, M. et al. Nature 448, 318-324 (2007); Maherali, N. et al. Cell Stem Cell 1, 55-70 (2007); Meissner, A., Wernig, M. & Jaenisch, R. Nature Biotechnol. 25, 1177-1181 (2007); Takahashi, K. et al. Cell 131, 861-872 (2007); Yu, J. et al. Science 318, 1917-1920 (2007); Nakagawa, M. et al. Nature Biotechnol. 26, 101-106 (2007); Wernig, M., Meissner, A., Cassady, J. P. & Jaenisch, R. Cell Stem Cell. 2, 10-12 (2008).

To address the safety issues that arise from target cell genomes harboring integrated exogenous sequences, a number of modified genetic protocols have been further developed and can be used according to the present invention. These protocols produce iPS cells with potentially reduced risks, and include non-integrating adenoviruses to deliver reprogramming genes (Stadtfeld, M., et al. (2008) Science 322, 945-949), transient transfection of reprogramming plasmids (Okita, K., et al. (2008) Science 322, 949-953), piggyBac transposition systems (Woltjen, K., et al. (2009). Nature 458, 766-770, Yusa et al. (2009) Nat. Methods 6:363-369, Kaji, K., et al. (2009) Nature 458, 771-775), Cre-excisable viruses (Soldner, F., et al. (2009) Cell 136, 964-977), and oriP/EBNA1-based episomal expression system (Yu, J., et al. (2009) Science DOI: 10.1126). Accordingly, in one embodiment, introduction of a polypeptide into a cell can comprise introduction of a polynucleotide comprising one or more expression cassettes into a cell and inducing expression, thereby introducing the polypeptides into the cell by transcription and translation from the expression cassette.

Alternatively, one or more proteins can simply be cultured in the presence of target cells under conditions to allow for introduction of the proteins into the cell. See, e.g., Zhou H et al., Cell Stem Cell. 2009 May 8; 4(5):381-4; WO/2009/117439. One can introduce an exogenous polypeptide (i.e., a protein provided from outside the cell and/or that is not produced by the cell) into the cell by a number of different methods that do not involve introduction of a polynucleotide encoding the polypeptide. Thus, in some embodiments, non-pluripotent cells are contacted with a GSK3 inhibitor (e.g., in the absence of an epigenetic modifier, or optionally in the presence of a histone H3k4 demethylase inhibitor or a H3K4 methylation activator) and one or more exogenous transcription factor proteins, e.g., one, two, or three of an Oct polypeptide, a Klf polypeptide, and a Myc polypeptide. In some embodiments, the exogenous proteins comprise the transcription factor polypeptide of interest linked (e.g., linked as a fusion protein or otherwise covalently or non-covalently linked) to a polypeptide that enhances the ability of the transcription factor to enter the cell (and in some embodiments the cell nucleus).

Examples of polypeptide sequences that enhance transport across membranes include, but are not limited to, the Drosophila homeoprotein antennapedia transcription protein (AntHD) (Joliot et al., New Biol. 3: 1121-34, 1991; Joliot et al., Proc. Natl. Acad. Sci. USA, 88: 1864-8, 1991; Le Roux et al., Proc. Natl. Acad. Sci. USA, 90: 9120-4, 1993), the herpes simplex virus structural protein VP22 (Elliott and O'Hare, Cell 88: 223-33, 1997); the HIV-1 transcriptional activator TAT protein (Green and Loewenstein, Cell 55: 1179-1188, 1988; Frankel and Pabo, Cell 55: 1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to an peptide sequence comprising at least 5-25 or more contiguous arginines or 5-25 or more arginines in a contiguous set of 30, 40, or 50 amino acids; including but not limited to an peptide having sufficient, e.g., at least 5, guanidino or amidino moieties); and commercially available Penetratin™ 1 peptide, and the Diatos Peptide Vectors (“DPVs”) of the Vectocell® platform available from Daitos S. A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes. A number of polypeptides capable of mediating introduction of associated molecules into a cell have been described previously and can be adapted to the present invention. See, e.g., Langel (2002) Cell Penetrating Peptides CRC Press, Pharmacology and Toxicology Series.

Exemplary polypeptide sequences that enhance transport across membranes include:

[symbol]

:
GSPPTAPTRSKTPAQGLARKLHFSTAPPNPDAPWTPRVAGFNKRVFRFS
PQTARRATTTRI; [symbol]
:
AGSGGAAVALLPAVLLALLAPGGEFA; [symbol]
:
AGSGGYARAAARQARAGGEFA; [symbol] [symbol]
:
RQIKIWFQGRRMKWKK; [symbol]
:
YGRKKRRQRRR; [symbol]
:
MVTVLFRRLRIRRACGPPRVRV; [symbol] [symbol]
:
AGYLLGKIGLKALAALAKKIL.

In some embodiments, the polypeptide that enhances transport across membranes is a peptide sequence comprising at least 5 or more contiguous or non-contiguous arginines (e.g., a 8-arginine peptide). In some embodiments, the polypeptide that enhances transport across membranes is a peptide sequence comprising at least 7 or more contiguous or non-contiguous arginines. For example, the polypeptide that enhances transport across membranes is a peptide sequence comprising 11 contiguous arginines, e.g., ESGGGGSPGRRRRRRRRRRR. As noted above, the arginines in the transport enhancing sequence need not all be contiguous. In some embodiments, the polyarginine (e.g., the contiguous or non-contiguous) region is at least 5, 8, 10, 12, 15, 20, or more amino acids long and has at least e.g., 40%, 50%, 60%, 70%, 80%, 90%, or more arginines.

An exogenous polypeptide can be introduced into cells by traditional methods such as lipofection, electroporation, calcium phosphate precipitation, particle bombardment and/or microinjection, or can be introduced into cells by a protein delivery agent. For example, the exogenous polypeptide can be introduced into cells by covalently or noncovalently attached lipids, e.g., by a covalently attached myristoyl group. Lipids used for lipofection are optionally excluded from cellular delivery modules in some embodiments. In some embodiments, the transcription factor polypeptides described herein are exogenously introduced as part of a liposome, or lipid cocktail such as commercially available Fugene6 and Lipofectamine). In another alternative, the transcription factor proteins can be microinjected or otherwise directly introduced into the target cell. In some embodiments, the transcription factor polypeptides are delivered into cells using Profect protein delivery reagents, e.g., Profect-P1 and Profect-P2 (Targeting Systems, El Cajon, Calif.), or using ProJect® transfection reagents (Pierce, Rockford Ill., USA). In some embodiments, the transcription factor polypeptides are delivered into cells using a single-wall nano tube (SWNT).

As discussed in the Examples of WO/2009/117439, incubation of cells with the transcription factor polypeptides of the invention for extended periods can be toxic to the cells. Therefore, the present invention provides for intermittent incubation of non-pluripotent mammalian cells with one or more of a Klf polypeptide and/or an Oct polypeptide (for example, Oct and Klf, no Myc or Sox) and/or a Myc polypeptide with intervening periods of incubation of the cells in the absence of the one or more polypeptides. In some embodiments, the cycle of incubation with and without the polypeptides can be repeated for 2, 3, 4, 5, 6, or more times and is performed for sufficient lengths of time (i.e., the incubations with and without proteins) to achieve the development of pluripotent cells. Various agents (e.g., MEK/ERK pathway inhibitor and/or TGFβ/ALK5 inhibitor and/or Rho GTPase/ROCK pathway inhibitor) can be included to improve efficiency of the method.

The various agents (e.g., GSK3 inhibitors, agents that promote H3K4 methylation or inhibit H3K4 demethylation, TGFβ receptor/ALK5 inhibitor, MEK/ERK pathway inhibitor, and/or Rho GTPase/ROCK inhibitor, etc.) can be contacted to non-pluripotent cells either prior to, simultaneous with, or after delivery of, programming transcription factors (for example, delivered via expression cassette or as proteins). For convenience, the day the reprogramming factors are delivered is designated “day 1”. In some embodiments, the inhibitors are contacted to cells in aggregate (i.e., as a “cocktail”) at about days 3-7 and continued for 7-14 days. Alternatively, in some embodiments, the cocktail is contacted to the cells at day 0 (i.e., a day before the preprogramming factors) and incubated for about 14-30 days.

The cell into which a protein of interest is introduced can be a mammalian cell. The cells can be human or non-human (e.g., primate, rat, mouse, rabbit, bovine, dog, cat, pig, etc.). The cell can be, e.g., in culture or in a tissue, fluid, etc. and/or from or in an organism. Cells that can be induced to pluripotency include, but are not limited to, keratinocyte cells, hair follicle cells, HUVEC (Human Umbilical Vein Endothelial Cells), cord blood cells, neural progenitor cells and fibroblasts.

### III. GSK-3 Inhibitors

The present invention provides for the use of Glycogen synthase kinase 3β (GSK3β, or, as used herein, “GSK-3”) inhibitors in reprogramming somatic animal cells into induced pluripotent cells.

Inhibitors of GSK3 can include antibodies that bind, dominant negative variants of, and siRNA, microRNA, antisense nucleic acids, and other polynucleotides that target GSK3. Specific examples of GSK3 inhibitors include, but are not limited to, Kenpaullone, 1-Azakenpaullone, CHIR99021, CHIR98014, AR-A014418 (see, e.g., Gould, et al., The International Journal of Neuropsychopharmacology 7:387-390 (2004)), CT 99021 (see, e.g., Wagman, Current Pharmaceutical Design 10:1105-1137 (2004)), CT 20026 (see, Wagman, supra), SB216763 (see, e.g., Martin, et al., Nature Immunology 6:777-784 (2005)), AR-A014418 (see, e.g., Noble, et al., PNAS 102:6990-6995 (2005)), lithium (see, e.g., Gould, et al., Pharmacological Research 48: 49-53 (2003)), SB 415286 (see, e.g., Frame, et al., Biochemical Journal 359:1-16 (2001)) and TDZD-8 (see, e.g., Chin, et al., Molecular Brain Research, 137(1-2):193-201 (2005)). Further exemplary GSK3 inhibitors available from Calbiochem (see, e.g., Dalton, et al., WO2008/094597, herein incorporated by reference), include but are not limited to BIO (2′Z,3′£)-6-Bromoindirubin-3′-oxime (GSK3 Inhibitor IX); BIO-Acetoxime (2′Z,3′£)-6-Bromoindirubin-3′-acetoxime (GSK3 Inhibitor X); (5-Methyl-1H-pyrazol-3-yl)-(2-phenylquinazolin-4-yl)amine (GSK3-Inhibitor XIII); Pyridocarbazole-cyclopenadienylruthenium complex (GSK3 Inhibitor XV); TDZD-8 4-Benzyl-2-methyl-1,2,4-thiadiazolidine-3,5-dione (GSK3beta Inhibitor I); 2-Thio(3-iodobenzyl)-5-(1-pyridyl)-[1,3,4]-oxadiazole (GSK3beta Inhibitor II); OTDZT 2,4-Dibenzyl-5-oxothiadiazolidine-3-thione (GSK3beta Inhibitor III); alpha-4-Dibromoacetophenone (GSK3beta Inhibitor VII); AR-AO 14418 N-(4-Methoxybenzyl)-N′-(5-nitro-1,3-thiazol-2-yl)urea (GSK-3beta Inhibitor VIII); 3-(1-(3-Hydroxypropyl)-1H-pyrrolo[2,3-b]pyridin-3-yl]-4-pyrazin-2-yl-pyrrole-2,5-dione (GSK-3beta Inhibitor XI); TWS1 19 pyrrolopyrimidine compound (GSK3beta Inhibitor XII); L803 H-KEAPPAPPQSpP-NH2 or its Myristoylated form (GSK3beta Inhibitor XIII); 2-Chloro-1-(4,5-dibromo-thiophen-2-yl)-ethanone (GSK3beta Inhibitor VI); AR-AO144-18; SB216763; and SB415286. Residues of GSK3b that interact with inhibitors have been identified. See, e.g., Bertrand et al., J. Mol Biol. 333(2): 393-407 (2003). Suitable GSK-3 beta inhibitors further include, but are not limited to, GF109203X (2-[1-(3-Dimethylaminopropyl)-1H-indol-3-yl]-3-(1H-indo1-3-yl)maleimide), RO318220 (2-[1-(3-(Amidinothio)propyl)-1H-indol-3-yl]-3-(1-methylindol-3-yl)maleimide methanesulfonate); SB216763 (3-([2,4-Dichlorophenyl)-4-(1-methyl-1H-indol-3yl)-1H-pyrrole-2,5-dione) (Santa Cruz Biotech, Santa Cruz, Calif.); SB415286 (3-[(3-Chloro-4-hydroxyphenyl)amino]-4-(2-nitrophenyl)-1H-pyrrole-2,5-dio-ne) (GlaxoSmithKline, London, United Kingdom); 4-Benzyl-2-methyl-1,2,4-thiadiazolidine-3,5-dione (“TDZD-8”) (Axxora, San Diego, Calif.); 2-Thio(3-iodobenzyl-5-(1-pyridyl)-[1,3,4]-oxadiazole (“TIBPO”) (Axxora, San Diego, Calif.); 2,4-Dibenzyl-5-oxothiadiazolidine-3-thione (“OTDZT”) (Axxora, San Diego, Calif.); and 4-(2-Amino-4-oxo-2-imidazolin-5-ylidene)-2-bromo-4,5,6,7-tetrahydropyrrol-o[2,3-c]azepin-8-one (“10Z-Hymenialdisine”) (Axxora, San Diego, Calif.). In addition, a number of monoclonal antibodies directed to GSK-3 beta are commercially available from Axxora. Other pharmacological inhibitors of GSK-3 beta are set forth in Meijer et al., “Pharmacological Inhibitors of Glyocogen Synthase Kinase 3, Trends in Pharmacological Sciences, Vol. 25, No. 9 (September 2004), which is incorporated by reference in its entirety.

GSK3 inhibitors can activate, for example, the Wnt/β-catenin pathway. Many of β-catenin downstream genes co-regulate pluripotency gene networks. For example, a GSK inhibitor activates cMyc expression as well as enhances its protein stability and transcriptional activity. Thus, in some embodiments, GSK3 inhibitors can be used to stimulate endogenous MYC polypeptide expression in a cell, thereby eliminating the need for MYC expression to induce pluripotency.

Those of skill will appreciate that the concentration of the GSK3 inhibitor will depend on which specific inhibitor is used. In certain embodiments, a combination of two or more different GSK3 inhibitors can be used.

### IV. Epigenetic Modifiers

As defined herein, the term “epigenetic modifier” refers to a methylation modifying agent (i.e., agents that induce methylation changes to DNA or histones) and/or an acetylation modifying agent (i.e., agents that induce acetylation changes to DNA or histones). In some embodiments, the methylation modifying agent is a DNA methylation inhibitor (e.g., a DNA methyltransferase (DNMT) inhibitor such as RG108)), histone methylation inhibitor and/or histone demethylation inhibitor. In some embodiments, the acetylation modifying agent is a histone deacetylase (HDAC) inhibitor (e.g., valproic acid or VPA), a histone acetyltransferase (HAT) inhibitor, histone deacetylase and histone acetyltransferase. In some embodiments, epigenetic modifiers are agents that inhibit methyltranferases or demethylases or agents that activate methyltranferases or demethylases. In some embodiment, the epigenetic modifier is an agent that inhibits histone H3K9 methylation or promotes H3K9 demethylation, e.g., a G9a histone methyltransferase such as BIX01294.

In some embodiments, the present invention provides for contacting a non-pluripotent animal cell with a GSK3 inhibitor (and optionally other agents as described herein) but not an agent that inhibits histone H3K9 methylation or promotes H3K9 demethylation. Examples of agents that inhibit histone H3K9 methylation include, e.g, BIX, and are described in e.g., WO/2009/117439. In some embodiments, the present invention provides for contacting a non-pluripotent animal cell with a GSK3 inhibitor (and optionally other agents as described herein) but not any epigenetic modifier.

In some embodiments, the present invention provides for contacting a non-pluripotent animal cell with a GSK3 inhibitor (and optionally other agents as described herein) and an inhibitor of histone H3K4 demethylation or an activator of H3K4 methylation. Examples of an inhibitor of histone H3K4 demethylation or an activator of H3K4 methylation include, e.g., inhibitors of lysine-specific demethylase 1. Exemplary inhibitors of lysine-specific demethylase 1 include, but are not limited to, parnate (tranylcypromine sulfate or equivalent salt) and phenelzine (Nardil, 2-phenylethylhydrazine). See, also, Huang et al., Proc Natl Acad Sci USA. 104(19): 8023-8028 (2007); Bi, X. et al., Bioorg. Med. Chem. Lett. 16:3229-3232 (2006); International Patent Application Nos. WO2007/021839 and WO2008/127734. The concentration of inhibitor of histone H3K4 demethylation or an activator of H3K4 methylation contacted to cells will depend on the specific agent used.

### V. Transformation

This invention employs routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

In some embodiments, the species of cell and protein to be expressed is the same. For example, if a mouse cell is used, a mouse ortholog is introduced into the cell. If a human cell is used, a human ortholog is introduced into the cell.

It will be appreciated that where two or more proteins are to be expressed in a cell, one or multiple expression cassettes can be used. For example, where one expression cassette expresses multiple polypeptides, a polycistronic expression cassette can be used.

### A. Plasmid Vectors

In certain embodiments, a plasmid vector is contemplated for use to transform a host cell. In general, plasmid vectors containing replicon and control sequences which are derived from species compatible with the host cell are used in connection with these hosts. The vector can carry a replication site, as well as marking sequences which are capable of providing phenotypic selection in transformed cells.

### B. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

i. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a ˜36 kb, linear, double-stranded DNA virus, allows substitution of large pieces of adenoviral DNA with foreign sequences up to 7 kb (Grunhaus et al., Seminar in Virology, 200(2):535-546, 1992)).

ii. AAV Vectors

The nucleic acid may be introduced into the cell using adenovirus assisted transfection. Increased transfection efficiencies have been reported in cell systems using adenovirus coupled systems (Kelleher and Vos, Biotechniques, 17(6):1110-7, 1994; Cotten et al., Proc Natl Acad Sci USA, 89(13):6094-6098, 1992; Curiel, Nat Immun, 13(2-3):141-64, 1994.). Adeno-associated virus (AAV) is an attractive vector system as it has a high frequency of integration and it can infect non-dividing cells, thus making it useful for delivery of genes into mammalian cells, for example, in tissue culture (Muzyczka, Curr Top Microbiol Immunol, 158:97-129, 1992) or in vivo. Details concerning the generation and use of rAAV vectors are described in U.S. Pat. Nos. 5,139,941 and 4,797,368, each incorporated herein by reference.

iii. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller et al., Am. J. Clin. Oncol., 15(3):216-221, 1992).

In order to construct a retroviral vector, a nucleic acid (e.g., one encoding gene of interest) is inserted into the viral genome in the place of certain viral sequences to produce a virus that is replication-defective. To produce virions, a packaging cell line containing the gag, pol, and env genes but without the LTR and packaging components is constructed (Mann et al., Cell, 33:153-159, 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986; Mann et al., Cell, 33:153-159, 1983). The media containing the recombinant retroviruses is then collected, optionally concentrated, and used for gene transfer. Retroviral vectors are able to infect a broad variety of cell types. However, integration and stable expression typically involves the division of host cells (Paskind et al., Virology, 67:242-248, 1975).

Lentiviruses are complex retroviruses, which, in addition to the common retroviral genes gag, pol, and env, contain other genes with regulatory or structural function. Lentiviral vectors are well known in the art (see, for example, Naldini et al., Science, 272(5259):263-267, 1996; Zufferey et al., Nat Biotechnol, 15(9):871-875, 1997; Blomer et al., J Virol., 71(9):6641-6649, 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

iv. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., Proc. Nat'l Acad. Sci. USA, 86:9079-9083, 1989). Using antibodies against major histocompatibility complex class I and class II antigens, they demonstrated the infection of a variety of human cells that bore those surface antigens with an ecotropic virus in vitro (Roux et al., 1989).

### C. Vector Delivery and Cell Transformation

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

### VI. Culture Media

The present invention provides a cell culture medium comprising mammalian non-pluripotent cells that do not express Sox2 (e.g., endogenously or heterologously), or are not in contact with a Sox2 polypeptide. Suitable mammalian non-pluripotent cells include ones that heterologously express a Myc polypeptide or are in contact with a Myc polypeptide. In some embodiments, a heterologous Myc polypeptide is not expressed in, or contacted to, the non-pluripotent cell.

The cell culture medium can comprise a GSK-3 inhibitor (and optionally other agents described herein) but not an agent that inhibits histone H3K9 methylation or promotes H3K9 demethylation. In some embodiments, the cell culture medium comprises a GSK-3 inhibitor and does not comprise any epigenetic modifiers (i.e., the cell culture medium is absent of an epigenetic modifier). Alternatively, the cell culture medium can comprise a GSK-3 inhibitor and an inhibitor of histone H3K4 demethylation or an activator of H3K4 methylation. Further, with or without an inhibitor of histone H3K4 demethylation or an activator of H3K4 methylation, the cell culture medium can comprise a Mek or Erk inhibitor and/or a TGFβ receptor inhibitor.

In some embodiments, the cell culture medium further comprises an exogenous Oct polypeptide or an exogenous Klf polypeptide or a combination thereof. For example, the cell culture medium comprises an exogenous Oct polypeptide and an exogenous Klf polypeptide. The amounts of GSK-3 inhibitor and/or the inhibitor of histone H3K4 demethylation and/or the activator of H3K4 methylation are in a range sufficient to induce mammalian non-pluripotent cells to pluripotency, e.g., induce at least 0.001% of the cells to pluripotency, e.g., in the presence of at least one of the exogenous polypeptides.

The cells according to the present invention can be human or non-human (e.g., primate, rat, mouse, rabbit, bovine, dog, cat, pig, etc.). Cells that can be induced to pluripotency include, but are not limited to, keratinocyte cells, hair follicle cells, HUVAC, cord blood cells, neural progenitor cells and fibroblasts.

Exemplary exogenous polypeptides that can be used in the cell culture medium of the present invention include Oct3/4, Klf1, Klf4, Klf5, c-Myc, N-Myc and L-Myc. In some embodiments, an Oct3/4 polypeptide is used. In some embodiments, a Klf4 polypeptide is used. A Klf1 and/or Klf5 polypeptide can replace a Klf4 polypeptide or can be used in combination with a Klf4 polypeptide. In some embodiments, a c-Myc, N-Myc and/or L-Myc is used. Mammalian (e.g., human, mouse or rat) transcription factors can be used in the present invention.

The exogenous polypeptides (e.g., the Oct polypeptide and/or the Klf polypeptide) used in the cell culture medium of the present invention can comprise a heterologous amino acid sequence that enhances transport across a cell membrane, as described above. For example, the exogenous polypeptides comprise a peptide sequence comprising at least 5 or more contiguous arginines or other sequences that enhance membrane transport.

### VII. Kits

The present invention provides a kit comprising an inhibitor of histone H3K4 demethylation or an activator of H3K4 methylation and a GSK-3 inhibitor. In some embodiments, the kit further comprises an exogenous Oct polypeptide or an exogenous Klf polypeptide or a combination thereof. For example, the kit comprises an Oct polypeptide and a Klf polypeptide. Further, the kit can comprise a Mek or Erk inhibitor and/or a TGFβ receptor inhibitor. The kit can also comprise a mammalian cell, e.g., a mammalian cell that does not express Sox2

Exemplary exogenous polypeptides that can be used in the kit of the present invention include Oct3/4, Klf1, Klf4, Klf5, c-Myc, N-Myc and L-Myc. In some embodiments, an Oct3/4 polypeptide is used. In some embodiments, a Klf4 polypeptide is used. A Klf1 and/or Klf5 polypeptide can replace a Klf4 polypeptide or can be used in combination with a Klf4 polypeptide. In some embodiments, a c-Myc, N-Myc and/or L-Myc is used. Mammalian (e.g., human, mouse or rat) transcription factors can be used in the present invention.

The polypeptides (e.g., the Oct polypeptide and/or the Klf polypeptide) can comprise a heterologous amino acid sequence that enhances transport across a cell membrane, as described above, e.g., a peptide sequence comprising at least 5 or more contiguous arginines.

Exemplary GSK-3 inhibitors include CHIR99021. Exemplary inhibitors of histone H3K4 demethylation include a monoamine oxidase inhibitor or a lysine-specific demethylase 1, e.g. parnate.

### EXAMPLES

The following examples are offered to illustrate, but not to limit the claimed invention.

### Example 1

Here, we report that a specific GSK-3 inhibitor, CHIR99021, could allow the reprogramming of both mouse and human somatic cells without Sox2 transgene. Our studies suggest that the GSK-3 inhibitor might have a general application to replace transcription factors in both mouse and human somatic cell reprogramming.

### Materials and Methods:

Cell Culture and Viral Transduction: MEFs were derived from 12952/SvPasCrlf and ROSA26^{+/-}/OG2^{+/-} mice according to the protocol reported on WiCell Research Institute website: “Introduction to human embryonic stem cell culture methods”. ROSA26^{+/-}/OG2^{+/-} transgenic mice carry GFP reporter gene under the control of the Oct4 promoter (Oct4-GFP) and the ubiquitously expressed neo/lacZ transgene [Do J T, Scholer H R, Stem Cells, 22:941-949 (2004)]. Animal experiments were performed according to the Animal Protection Guidelines of the Max Planck Institute for Biomolecular Research, Germany. MEFs were transduced by Oct4, Klf4 and Sox2 three factors, or two-factor combinations of the pMXs-based retroviruses encoding mouse Oct4, Klf4 and Sox2 (Addgene) as previously described [Takahashi K, Yamanaka S, Cell, 126:663-676 (2006)]. Twenty four hours later, transduced MEFs were seeded in 6-well plate and incubated with mESC growth medium: Knockout™ DMEM, 7% ES Cell-Qualified fetal bovine serum, 10% Knockout Serum Replacement, 1% Glutamax, 1% Non-essential amino acids, 1% penicillin/streptomycin, 0.1 mM β-mercaptoethanol and 10³ U/ml mLIF (Millipore). MEFs transduced with Oct4/Klf4/Sox2 (1×10⁴ cells/well together with 10⁵ cells/well CF1 feeders in 6-well plates) were then treated with GSK-3 inhibitor CHIR99021 (Stemgent) for two weeks, and EGFP positive colonies were picked up at the third week after treatment. MEFs transduced with Oct4/Klf4 (1×10⁵ cells/well in 6-well plates) were treated with 10 μM CHIR99021 for four weeks, GFP positive colonies were picked up and expanded at the fourth to fifth week after treatment.

Neonatal Human Epidermal Keratinocytes (NHEKs, Lonza) were cultured and transduced with two-factor combinations of lentiviruses encoding human Oct4, Sox2 (pSin-EF2-Puro-based) and mouse Klf4 (pLOVE-based) as previously described [Yu et al., Science, 318:1917-1920 (2007); Blelloch et al., Generation of Induced Pluripotent Stem Cells in the Absence of Drug Selection, 1:245-247 (2007)]. Lentiviral vectors were obtained from Addgene. Twenty four hours later, 1×10-transduced NHEKs were seeded on the irradiated X-ray inactivated CF1 MEF feeder cells in a 100 mm dish by keratinocyte medium (Lonza). One week after, the media was changed to human ES cell medium: DMEM/F12, 20% Knockout serum replacement, 1% Glutamax, 1% Non-essential amino acids, 1% penicillin/streptomycin, 0.1 mM β-mercaptoethanol and 100 ng/ml bFGF and treated with GSK-3 inhibitor CHIR99021 (Stemgent) (10 μM) alone or combined with valproic acid (0.5˜2 mM), BIX-01294 (Stemgent) (1˜2 μM), RG108 (Stemgent) (1˜5 μM), Parnate (Sigma) (2˜μM), PD0325901 (Stemgent) (0.5 μM) and SB431542 (Tocris) (2 μM). The media containing above small molecule combinations were changed every day. Two week after treatment, the cells were sub-cultured (1:1) on new feeder cells (PD0325901 and SB431542 were only used in the first two-week treatment). After another two weeks, the small molecules were removed and the cells were stained with Alexa Fluor 555-conjugated Mouse anti-Human TRA-1-81 antibody (BD Pharmingen). The positive colonies were marked and picked up for expansion on feeder cells in human ES cell medium about 7 weeks after transduction. The human iPSCs were sub-cultured regularly by Accutase (Chemicon). All cell culture products were from Invitrogen/Gibco BRL except where mentioned.

Cytochemistry and Immunofluorescence Assay: Alkaline Phosphatase staining was performed according to the manufacturer's protocol using the Alkaline Phosphatase Detection Kit (Millipore). For immunofluorescence assay, cells were fixed in 4% paraformaldehyde for 10 minutes and washed three times with PBS containing 0.1% Triton X-100 (Sigma-Aldrich). The fixed cells were then incubated in blocking buffer, 0.1% Triton X-100 and 10% normal donkey serum (Jackson ImmunoResearch Laboratories Inc) in PBS (Invitrogen/Gibco BRL), for 30 min at room temperature. The cells were then incubated with primary antibody overnight at 4° C. in blocking buffer. The day after, cells were washed with PBS and incubated with secondary antibody in PBS containing 0.1% Triton X-100 for one hour at room temperature. Mouse anti-Oct4 antibody (1:250) (Santa Cruz Biotechnology), rabbit anti-Sox2 antibody (1:2000) (Chemicon), mouse anti-SSEA1 antibody (1:250) (Santa Cruz Biotechnology), rabbit anti-Nanog antibody (1:250) (Abcam), rat anti-SSEA3 antibody (1:1000) (Chemicon), mouse anti-SSEA4 antibody (1:1000) (Chemicon), mouse anti-TRA-1-81 antibody (1:1000) (Chemicon), goat anti-Sox17 (1:200) (R&D), mouse anti-βIII-Tubulin (Tuj1) antibody (1:1000) (Covance Research Products), rabbit anti-Brachyury antibody (1:200) (Santa Cruz) were used as primary antibodies. Secondary antibodies were Alexa Fluor 486/555 donkey anti-mouse, anti-rat, anti-goat or anti-rabbit IgG (1:500) (Invitrogen). Nuclei were visualized by DAPI staining (Sigma-Aldrich). Images were captured using a Nikon Eclipse TE2000-U microscope.

Differentiation of iPSCs in Vitro: The in vitro differentiation of miPSCs-OK and hiPSCs-OK was carried out by the standard embryoid body (EB) differentiation method. The iPSCs were dissociated by either 0.05% Trypsin-EDTA (miPSCs-OK) or Accutase (hiPSCs-OK), and then cultured in ultra-low attachment 100-mm dish in DMEM medium supplemented with 10% FBS to form EBs. The medium was changed every other day. One week later, the EBs were harvested and transferred into Matrigel-coated 6-well plate in DMEM medium with 10% FBS. Three to seven day later, the cells were fixed for immunocytochemistry analysis.

PCR analysis: To detect the expression of pluripotency genes by MEFs and NHEKs that were treated with small molecules, untransduced MEFs and NHEKs were treated for three days in mESC growth medium with 10 μM CHIR99021 or in hES cell medium with either combination of 10 μM CHIR99021 and 2 μM Parnate or combination of 10 μM CHIR99021, 2 μM Parnate, 0.5 μM PD0325901 and 2 μM SB431542. For the semi-quantitative and quantitative RT-PCR analyses, RNA was extracted from miPSCs-OK, hiPSCs-OK, MEFs, treated MEFs and treated NHEKs using the RNeasy Plus Mini Kit in combination with QIAshredder (Qiagene). Reverse transcription was performed with 1 μg RNA using iScript™ cDNA Synthesis Kit (BioRad). The expression of pluripotent markers by miPSCs-OK was analyzed by RT-PCR using Platinum PCR SuperMix (Invitrogen). The primers for the endogenous Oct4, Sox2, Klf4 and Nanog were as reported [Takahashi K, Yamanaka S, Cell, 126:663-676 (2006)]. Amplification of viral transduced genes was done using the gene specific forward primers (Klf4: 5′-GCG AAC TCA CAC AGG CGA GAA ACC-3′; Sox2: 5′-GGT TAC CTC TTC CTC CCA CTC CAG-3′ and Oct4: 5′-TTG GGC TAG AGA AGG ATG TGG TTC-3′) and common reverse primer pMXs-L3205 (5′-CCC TTT TTC TGG AGA CTA AAT AAA-3′) [Takahashi et al, Cell, 131:861-872 (2007)]. The RT-PCR was performed in 30 (amplification of pluripotent markers) or 35 (amplification of viral transduced genes) cycles (94° C. for 30s, annealing temperature for 30s, and 72° C. for 30s). Real-time PCR was carried out using iQ SYBR Green Supermix (BioRad). The primers for the human endogenous Oct4, total Oct4, endogenous Sox2, total Sox2, Nanog, Klf4, GDF-3 and Cripto were as reported [Yu et al., Science, 318:1917-1920 (2007); Aasen et al., Nat Biotechnol., 26:1276-1284 (2008); Mateizel et al., Hum Reprod., 21:503-511 (2006)4, 26, 27]. The primer for viral Klf4 was 5′-CAC CTT GCC TTA CAC ATG AAG AGG-3′ and 5′-CGT AGA ATC GAG ACC GAG GAG A-3′. The primer for FGF-4 was 5′-GAC ACC CGC GAC AGC CT -3′ and 5′-TCA CCA CGC CCC GCT-3′. The expression of genes of interest was normalized to that of GAPDH in all samples.

Genomic DNA was extracted from miPSCs-OK using DNeasy Blood & Tissue Kit (QIAGEN). In order to analyze the viral integration in miPSCs-OK, the genomic DNA of miPSCs-OK was subjected to PCR analysis using the same primers employed to amplify the viral transduced genes in the RT-PCR experiments. For the methylation analysis of Oct4 promoter by bisulfite-sequencing, DNA samples from hiPSC-OK were isolated using the Non Organic DNA Isolation Kit (Millipore) and were then treated with the EZ DNA Methylation-Gold Kit (Zymo Research Corp., Orange, Calif.). The treated DNA samples were then used as templates to amplify targets of interest. Primers used for the amplification of the Oct4 promoter fragment (406 bp, from −2192˜−1786) were 5′-GGA TGT TAT TAA GAT GAA GAT AGT TGG-3′ and 5′-CCT AAA CTC CCC TTC AAA ATC TAT T-3′ [Deb-Rinker et al., J. Biol. Chem., 280:6257-6260 (2005)]. The resulting fragments were cloned using the TOPO TA Cloning Kit for sequencing (Invitrogen) and sequenced.

Aggregation of iPSCs with zona-free embryos: miPSCs-OK were aggregated with denuded post-compacted eight-cell stage embryos to obtain aggregate chimeras. Eight-cell embryos (B6C3F1) were flushed from females at 2.5 dpc and cultured in microdrops of KSOM medium (10% FCS) under mineral oil. Clumps of iPSCs (10-20 cells) after short treatment of trypsin were chosen and transferred into microdrops containing zona-free eight-cell embryos. Eight-cell embryos aggregated with iPSCs were cultured overnight at 37° C., 5% CO₂. Aggregated blastocysts that developed from eight-cell stage were transferred into one uterine horn of a 2.5 dpc pseudopregnant recipient. The recipient mice were sacrificed at ED 13.5 day. The embryos were analyzed by x-gal staining to reveal the contribution of iPS cells.

Teratoma Formation: Three to five million hiPSC-OK (passage 8, clone 1) were injected under the kidney capsule of SCID mice (n=3). After 6-8 weeks, the neoplasm was removed and then histologically analyzed.

### RESULTS

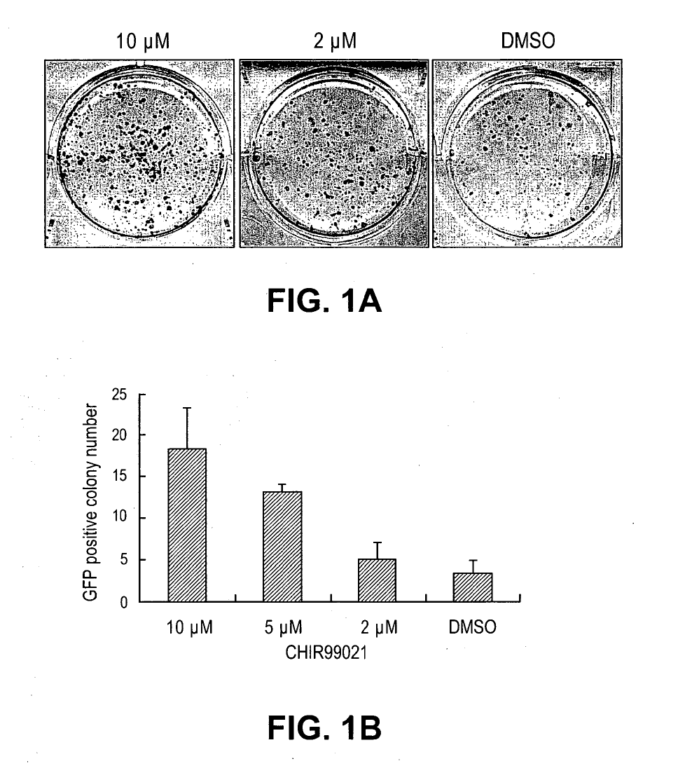

CHIR99021 can significantly promote the reprogramming of MEFs transduced by Oct4, Sox2 and Klf4. It had been shown that Oct4/Sox2/Klf4-infected MEFs could be reprogrammed into pluripotent state with higher efficiency when cultured under Wnt3a-conditioned medium [Marson et al., Cell Stem Cell, 3:132-135 (2008)]. However, small molecule activators of Wnt signaling pathway were not found to have similar effects. Combination of CHIR99021, a GSK-3 inhibitor which can activate Wnt signaling pathway, with PD0325901, a MEK inhibitor, was shown to promote partially reprogrammed iPSCs to full pluripotency [Silva et al., PLoS Biol., 6:e253 (2008)]. Concurrent with those studies, we found that CHIR99021 could significantly promote reprogramming of murine fibroblasts. Treating Oct4/Sox2/Klf4 transduced MEFs with CHIR99021 for two weeks significantly increased the number of alkaline phosphase (AP)-positive mESC-like colonies in a dose-dependent manner (AP staining was done at the third week after treatment) (FIG. 1A). CHIR99021 treatment of Oct4/Sox2/Klf4-transduced MEFs (ROSA26^{+/-}/OG2^{+/-}), which express GFP under the control of Oct4 promoter and also ubiquitously LacZ, also increased the number of GFP-positive colonies, which could be observed as early as two weeks after treatment. CHIR99021 showed the greatest effects at about 10 μM, which increase efficiency from 0.03-0.08% to 0.2-0.4% of transduced MEFs. (FIG. 1B). Our results therefore suggest that CHIR99021 can significantly improve reprogramming efficiency of MEFs transduced with Oct4, Sox2, and Klf4. These mouse iPS cell colonies could be stably expanded under conventional mESC growth condition and express typical pluripotency markers, such as AP, Oct4, Sox2, Nanog, SSEA1 by cytochemistry and immunostaining

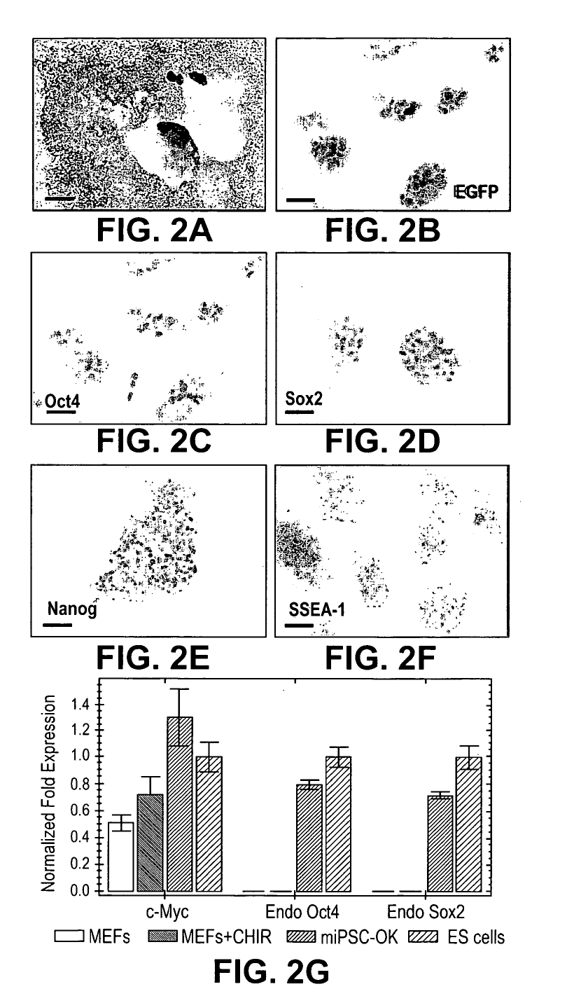

CHIR99021 enabled the reprogramming of MEFs transduced by Oct4/Klf4. We had previously identified BIX01294, a small molecule inhibitor of a histone methyltransferase G9a, which enabled reprogramming of both mouse NPCs and MEFs infected by only Oct4 and Klf4 [Shi et al., Cell Stem Cell., 2:525-528 (2008); Shi et al., Cell Stem Cell, 3:568-574 (2008)]. We then investigated whether iPS cells could be generated from MEFs with fewer reprogramming factors in the presence of CHIR99021. 0G2 MEFs transduced with different two-factor combinations (Oct4/Klf4, Oct4/Sox2 and Sox2/Klf4) were treated with 10 μM CHIR99021. GFP positive iPS cell colonies were identified only when MEFs were transduced with the combination of Oct4 and Klf4, but not with any other combination. On average, about six GFP positive colonies were identified out of 10⁵ OG2 MEFs 4˜5 weeks after Oct4/Klf4 transduction and CHIR99021 treatment. Stable iPS cell lines (miPSC-OK) were established by picking up the GFP positive colonies (FIG. 2A, 2B). Immunocytochemistry revealed that miPSC-OK express typical pluripotency markers, such as Oct4, Sox2, Nanog and SSEA-1 (FIG. 2C-F). MEFs do not express Sox2 endogenously, and real-time PCR analysis revealed that CHIR99021 treatment did not induce the expression of Sox2 and Oct4 in MEFs (FIG. 2G). Therefore, the mechanisms by which CHIR99021 promotes the reprogramming of MEFs transduced by Oct4/Klf4 are independent of direct Sox2 induction.

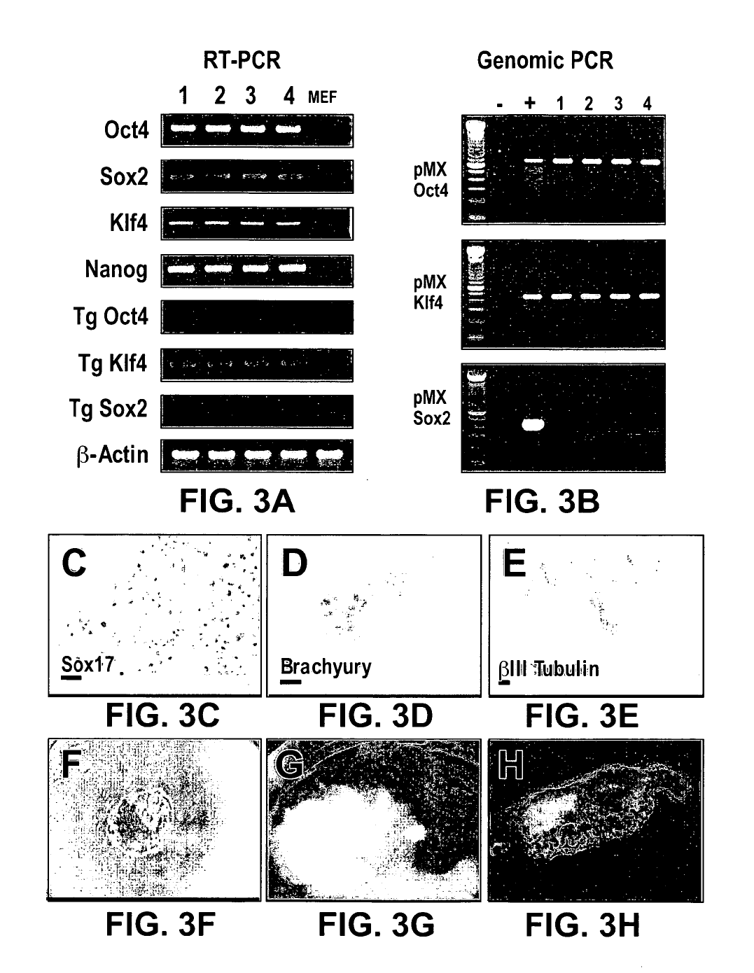

RT-PCR analysis confirmed the reactivation and expression of the endogenous mouse Oct4, Sox2, Nanog, and Klf4 (FIG. 3A). By using the specific primers for transgenes, RT-PCR analysis revealed that the viral genes were largely silenced (FIG. 3A). PCR of genomic DNA of miPSC-OK confirmed the integration of retroviral Oct4 and Klf4, but no other reprogramming genes (FIG. 3B). To examine the developmental potential of miPSC-OK, in vitro differentiation assay was preformed. Immunostaining showed miPSC-OK could differentiate into endoderm (Sox17), mesoderm (brachyury) and neuroectoderm (βIII-tubulin) derivatives under the standard embryoid body differentiation methods (FIG. 3C-E). Most importantly, miPSC-OK (clonel and clone2) could efficiently incorporate into the inner cell mass (ICM) of blastocysts after aggregation with 8-cell embryos, led to mid-gestational chimerism (ED13.5) after the aggregated embryos were transplanted into mice, and contributed to germline cells in vivo (FIG. 3F-H). Twenty transplanted embryos aggregated with miPSC-OK (clone 1) were allowed to be born. However, no chimeric mice are identified from the living pups. These in vitro and in vivo characterizations confirm that the miPSC-OK are molecularly, morphologically, and functionally similar to the original four-factor iPS cells and the mouse ESCs.

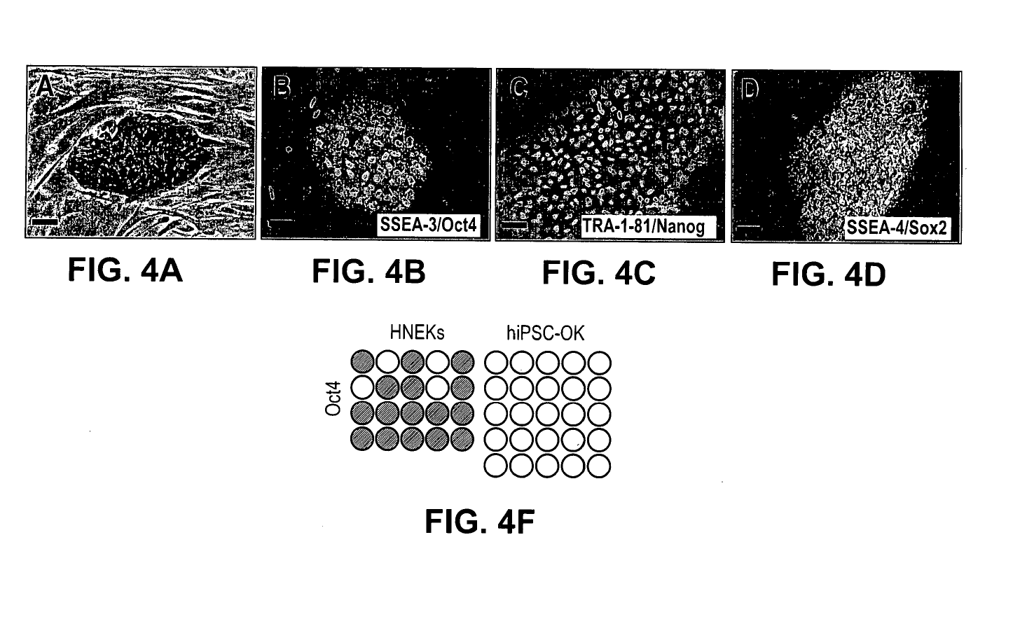

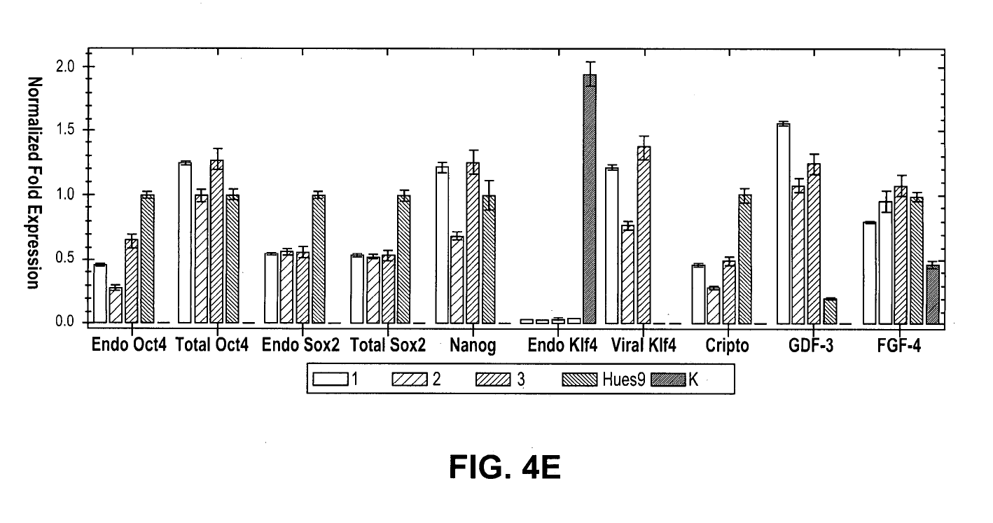

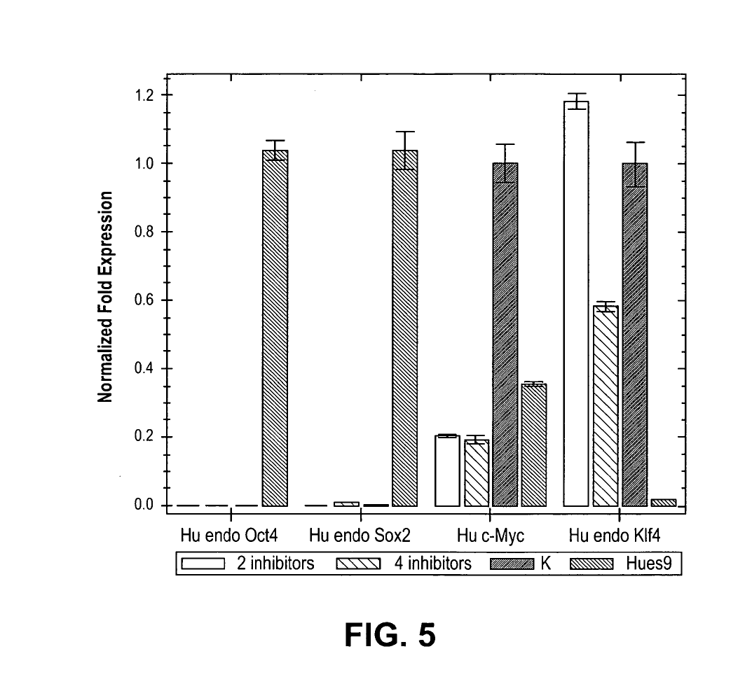

CHIR99021 enabled the reprogramming of human neonatal keratinocytes transduced with Oct4/Klf4 when combined with Parnate. We next investigated whether human iPS cells could be generated with fewer transcription factors in the presence of CHIR99021 and/or direct epigenetic modifiers including inhibitors of DNA methyltransferase (RG108), histone methyltransferase (BIX01294), histone deacetylase (valproic acid/VPA) and lysine-specific demethylase 1 (Parnate). To this end, we selected primary human neonatal epidermal keratinocytes (HNEKs), concurrent with recent studies suggesting that keratinocytes transduced with four factors could be reprogrammed into iPS cells more efficiently and rapidly in comparison to other somatic cell types [Aasen et al., Nat Biotechnol., 26:1276-1284 (2008)]. Primary keratinocytes were transduced with different two-factor combinations (Oct4/Klf4, Oct4/Sox2 and Sox2/Klf4), treated with CHIR99021 alone, or combined with epigenetic modifiers, and then stained with the human pluripotency cell-surface marker TRA-1-81 5 weeks post-infection. Tra-1-81 positive human ESC-like colonies could only be identified from culture infected by Oct4 and Klf4 in the presence of CHIR99021 and Parnate. On average, about two Tra-1-81 positive colonies could be identified out of 10⁵ transduced HNEKs, which was at lease 100 times less efficient than 4-factor transduced keratinocytes. Stable human iPS cells could be established and long-term expanded by picking up these colonies (named hiPSC-OK). In addition, we have also found that combined treatment using inhibitors of MEK (PD0325901) and TGFβ receptor (SB431542) could improve the reprogramming efficiency of human fibroblasts transduced by Oct4/Sox2/Klf4/c-Myc (unpublished data). By using CHIR99021 (10 μM) and Parnate (2 μM) as the basal condition, addition of PD0325901 (0.5 μM) and SB431542 (2 μM) could further increase the TRA-1-81 positive colonies from human keratinocytes transduced with Oct4/Klf4 (about 5-10 Tra-1-81 positive colonies could be identified out of 10⁵ transduced HNEKs), but the detailed mechanisms underlying this observation still need to be revealed. Nine TRA-1-81 positive colonies were expanded, and four stable human iPS cells (hiPSC-OK), one from CHIR99021 and Parnate condition (hiPSC-OK 1) and another three from CHIR99021/Parnate plus PD0325901/SB431542 condition (hiPSC-OK 2-4), were further studied and long-term cultured for over 20 passages. hiPSC-OK express typical pluripotency markers, such as AP, Oct4, Sox2, Nanog, TRA-1-81, SSEA3 and SSEA-4 (FIG. 4A-D). Real-time PCR analysis confirmed expression of the endogenous human Oct4, Sox2, Nanog, Cripto, GDF-3 and FGF4 (FIG. 4E). Although the viral Oct4 and Klf4 expression was not completely silenced, bisulfite sequencing analysis revealed that the Oct4 promoter of hiPSC-OK is largely demethylated (FIG. 4F). Similar to the CHIR99021 treatment of MEFs, real-time PCR analysis indicated neither CHIR99021/Parnate (2 inhibitors) nor CHIR99021/Parnate/PD0325901/SB431542 (4 inhibitors) treatment induced the expression of Sox2 and Oct4 in keratinocytes immediately (FIG. 5). The terminal differentiation of keratinocytes induced by the human ES cell culture media may result in the significant down-regulation of c-Myc expression after treatment (FIG. 5).

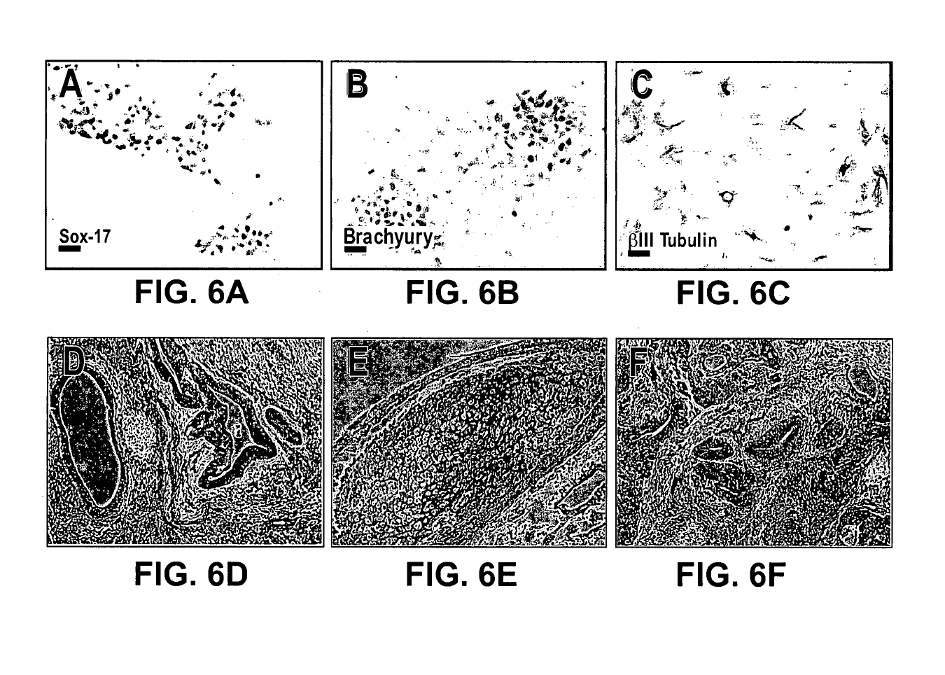

To examine developmental potentials of hiPSC-OK, in vitro differentiation assays were preformed. Immunostaining confirmed that hiPSC-OK could differentiate into endoderm (Sox17), mesoderm (brachyury) and neuroectoderm (βIII-tubulin) (FIG. 6A-C) derivatives in vitro. Furthermore, after transplanted into the SCID mice, hiPSC-OK formed teratoma consisting of representative derivatives of all three germ layers including epithelial tube structure (endoderm) (FIG. 6D), cartilage-like structure (mesoderm) (FIG. 6E) and neuroepithelium-like structure (ectoderm) (FIG. 6F). These in vitro and in vivo characterizations confirm that the human iPS cells generated by Oct4 and Klf4 viral transduction closely resemble human ES cells in terms of typical pluripotency marker expression and differentiation potential.

### DISCUSSION

Reprogramming is a very slow and inefficient process. Such low efficiency and slow kinetics also present “hidden” risks in iPSCs, such as accumulated and selected subtle genetic and epigenetic abnormalities. Consequently, it is highly desirable to identify new conditions/small molecules that can promote reprogramming and/or replace certain factors.

In the present study we reported that the GSK-3 inhibitor CHIR99021 can significantly improve the reprogramming efficiency of MEFs transduced by Oct4/Sox2/Klf4, and also enable the reprogramming of MEFs transduced by only Oct4 and Klf4. When combined with Parnate, CHIR99021 can result in the reprogramming of human primary keratinocytes transduced with only Oct4 and Klf4 as well. Although previous studies showed that the activation of Wnt signaling promote somatic cell reprogramming, this study is the first report to show GSK-3 inhibitor could allow the reprogramming of both mouse and human somatic cell without Sox2. Recently it is reported that the target genes co-bounded by Oct4, Sox2 and Klf4 in ES cells showed a lower histone H3 lysine 4 (H3K4) trimethylation enrichment in partially reprogrammed cells than in ES/iPS cells, and this low histone H3K4 trimethylation may result in the lack of binding of many important regulators of pluripotency by Oct4, Sox2 and Klf4 [Sridharan et al., Cell, 136:364-377 (2009)]. Parnate, a monoamine oxidase inhibitor used as an antidepressant drug, showed potent inhibitory effect on lysine-specific demethylase 1 and inhibiting the H3K4 demethylation, but does not influence the acetylation of H3K9/K14 [Mimasu et al., Biochem Biophys Res Commun., 366:15-22 (2008)]. Parnate may facilitate the full reprogramming of HNEK transduced with only Oct4 and Klf4 by inhibiting H3K4 demethylation.

Particularly, this is also the first time to generate human iPS cells from somatic cells without exogenous Sox2 expression. Both Oct4 and Sox2 are critical regulators in human/mouse ES cell pluripotency and also the only common reprogramming factors used for generation of human iPS cells. Replacement of Sox2 in human cell reprogramming represents an important step toward identifying a chemically defined condition that could allow reprogramming human somatic cells by Oct4 only or without the forced expression of any exogenous factor. As HNEKs express Klf4 endogenously, it would be conceivable that HNEKs could perhaps be fully reprogrammed with only Oct4 transduction, but so far it was not achieved for unknown reasons. When Oct4 transduced HNEKs were treated under the same chemical condition, some human ES cell-like colonies were observed. After picking up these colonies, stable lines were established which could be long-term cultured under conventional human ES cell media. However, these cells are negative to AP staining, and expression of other pluripotency markers, such as Nanog and Sox2 could not be detected by immunostaining

Our studies underscore the unique advantage of the chemical approach for improving reprogramming that may ultimately allow the generation of iPS cells or multipotent tissue-specific cells in completely chemically defined conditions without any permanent genetic modification. Ultimately, a completely chemically defined condition for efficient reprogramming of somatic cells would be highly favorable for various iPS cell applications.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of producing an induced pluripotent stem cell from a mammalian non-pluripotent cell that does not endogenously or heterologously express Sox2 and is not in contact with a Sox2 polypeptide, the method comprising,
introducing at least an Oct polypeptide and a Klf polypeptide into the non-pluripotent cell; and
contacting the non-pluripotent cell with:
a GSK-3 inhibitor in the absence of an epigenetic modifier, or
a GSK-3 inhibitor and an inhibitor of histone H3K4 demethylation or an activator of H3K4 methylation,
under conditions to generate a pluripotent cell, thereby producing an induced pluripotent stem cell from the mammalian non-pluripotent cell.

2. The method of claim 1, wherein the Oct polypeptide comprises an Oct3/4 polypeptide.

3. The method of claim 1, wherein the Klf polypeptide comprises a Klf4 polypeptide.

4. The method of claim 1, wherein the GSK-3 inhibitor is CHIR99021.

5. The method of claim 1, wherein the Oct polypeptide comprises a human or mouse Oct polypeptide and the Klf polypeptide comprises a human or mouse Klf polypeptide.

6. The method of claim 1, wherein the introducing step comprises contacting the cells with the Oct polypeptide and the Klf polypeptide under conditions to allow for entry of the Oct polypeptide and the Klf polypeptide into the cell.

7. The method of claim 6, wherein:
the Oct polypeptide comprises a heterologous amino acid sequence that enhances transport across a cell membrane; and
the Klf polypeptide comprises a heterologous amino acid sequence that enhances transport across a cell membrane.

8. The method of claim 1, wherein the introducing step comprises contacting the cells with a polynucleotide encoding the Oct polypeptide and a polynucleotide encoding the Klf polypeptide under conditions to allow for (1) entry of the polynucleotide into the cell and (2) expression of the Oct polypeptide and the Klf polypeptide from the introduced polynucleotide.

9. The method of claim 1, wherein a heterologous Myc polypeptide is not introduced into, or contacted to, the non-pluripotent cell.

10. (canceled)

11. The method of claim 1, wherein the contacting step further comprises contacting the cell with a Mek inhibitor and/or a TGFβ receptor inhibitor.

12. The method of claim 1, wherein the inhibitor of histone H3K4 demethylation is a monoamine oxidase inhibitor.

13. The method of claim 12, wherein the inhibitor of histone H3K4 demethylation is parnate.

14. The method of claim 1, wherein the non-pluripotent cell is selected from a fibroblast and a keratinocyte.

15. The method of claim 1, wherein the cell is human.

16. (canceled)

17. A cell culture medium comprising:
mammalian non-pluripotent cells that are not in contact with or express, Sox2;
an exogenous Oct polypeptide and an exogenous Klf polypeptide; and
a sufficient amount of:
a GSK-3 inhibitor in the absence of an epigenetic modifier, or
a GSK-3 inhibitor and an inhibitor of histone H3K4 demethylation or an activator of H3K4 methylation,
to induce at least 0.001% of the cells to pluripotency in the presence of the exogenous polypeptides.

18. The cell culture medium of claim 17, wherein the culture does not include an agent that inhibits histone H3K9 methylation or promotes H3K9 demethylation.

19. The cell culture medium of claim 17, wherein the Oct polypeptide comprises an Oct3/4 polypeptide.

20. The cell culture medium of claim 17, wherein the Klf polypeptide comprises a Klf4 polypeptide.

21. The cell culture medium of claim 17, wherein the GSK-3 inhibitor is CHIR99021.

22-31. (canceled)

32. A kit comprising:
an inhibitor of histone H3K4 demethylation or an activator of H3K4 methylation; and
a GSK-3 inhibitor.

33-41. (canceled)
